(19) United States
(12) Patent Application Publication
(10) Pub. No.: US 2003/0060483 A1
(43) Pub. Date: Mar. 27, 2003

(54) Method of increasing pharmaceutical market

(76) Inventor: Charles E. Day, Leitchfield, KY (US)

Correspondence Address: Dr. Charles E. Day, 1434 Sunbeam Road, Leitchfield, KY, 42754, US

(21) Appl. No.: 09/961,540

(22) Filed: Sep. 24, 2001

(51) Int. Cl. A61K 31/47; A01N 43/42

(52) U.S. Cl. 514/310

(57) ABSTRACT

Disclosed is a method of increasing the market for an existing pharmaceutical including the step of making a reformulated pharmaceutical by combining the existing pharmaceutical with a nondegradable synthetic polymer not substantially absorbed from the human alimentary canal.

### FIELD OF THE INVENTION

The invention relates to a method for increasing pharmaceutical sales.

### BACKGROUND AND DESCRIPTION OF THE INVENTION

The pharmaceutical industry produces tens of billions of dollars of revenue each year. Considerable efforts are expended, and expenditures incurred, in increasing the market for pharmaceutical products. These efforts include not only traditional marketing efforts but also research and development to reformulate existing drugs and even legal and legislative efforts to extend the patent term of a drug by as little as a few days, sometimes generating tens of millions of dollars of additional revenue to the patentee.

There are limits, however, to the effectiveness of these efforts. While traditional marketing efforts are limited primarily by the marketers' budget and creativity—and the elasticity of demand—reformulation R&D efforts tend to be focused on altering the dosage form to impart a favorable property to the formulation, or on increasing the patent term of the drug by chemically altering an active component or making a novel and nonobvious change in the dosage form. It is a truism that most contemplated changes do not meet this latter standard. Legal and legislative efforts have primarily to do with making sure as long a patent term as possible is secured for every drug in a company's portfolio. At present it is highly unlikely that more than a few years can be added to a U.S. patent term in compensation for a delay in FDA approval of a patented drug, absent Congressional intervention.

Hence there is a longstanding need for a method of increasing the market for an existing pharmaceutical composition that depends on something other than lobbying or litigation or minor changes in a drug's dosage form.

The present invention addresses this need. The invention relates to a method of increasing the market for an existing pharmaceutical composition including the step of reformulating that pharmaceutical by combining the existing pharmaceutical with a nondegradable synthetic polymer that is not substantially absorbed from the human alimentary canal.

In the present context, “market” means total sales. For example, a market is increased by selling a larger number of units per fixed interval of time, or by selling a fixed number of units for a longer interval of time. Lengthening the patent term of a drug and widening the geographic scope of a drug's sales are two ways in which market is increased. Also, enhancing the safety or efficacy of a drug is a way to increase a drug's market. Furthermore, a way to increase a drug's market is to reformulate the drug's previous dosage form by use of a synthetic polymer to replace materials such as gelatin, found objectionable and hence shunned by certain consumers; that is, the previous dosage form is not consumed by those who would otherwise be consumers of the drug, and these same consumers prefer and hence consume the reformulated dosage form.

In the present context, a “pharmaceutical” is a composition that is useful as a pharmaceutical or therapeutic agent, whether by itself or in combination with one or more other compositions.

In the present context, “nondegradable” means not being substantially hydrolyzed or otherwise subject to substantial covalent modification during transit through the human alimentary canal.

In the present context, “synthetic” means produced by human or mechanical effort via chemical synthesis, i.e., not known to occur in nature.

In the present context, “substantial” and “substantially” have their ordinary meanings, in that, for example, hydrolysis of more than 50% of the amide bonds in a protein would be substantial covalent modification of the protein.

In the present context, “consume” means ingest, purchase, or cause to be ingested or purchased.

### EXAMPLES

It is known in the art that diarrhea is an adverse effect experienced with some frequency by patients administered any of several members of the class of selective serotonin reuptake inhibitors. The market for fluoxetine, a member of that class, is increased by combining an amount of the copolymer of 1-(3-aminopropyl)imidazole and epichlorohydrin (also called “A2497”; disclosed in U.S. Pat. No. 5,900,233 to Day) therapeutically effective for the treatment, lessening, or prevention of diarrhea and an amount of fluoxetine therapeutically effective for the treatment, lessening, or prevention of depression. The resultant combination is consumed by individuals at risk of diarrhea due to fluoxetine administration who otherwise consume fewer units of fluoxetine. Also, the patent term of the combination extends beyond that of fluoxetine. Sales of fluoxetine increase and the market for fluoxetine is increased.

In another example, the market for fluoxetine is increased by combining from about 0.5 g to about 20 g A-2497 and from about 10 mg to about 80 mg fluoxetine. The resultant combination is consumed by individuals at risk of diarrhea due to fluoxetine administration who otherwise consume fewer units of fluoxetine. Also, the patent term of the combination extends beyond that of fluoxetine. Sales of fluoxetine increase and the market for fluoxetine is increased.

In another example, the market for paroxetine is increased by combining an amount of A-2497 therapeutically effective for the treatment, lessening, or prevention of diarrhea and an amount of paroxetine therapeutically effective for the treatment, lessening, or prevention of depression. The resultant combination is consumed by individuals at risk of diarrhea due to paroxetine administration who otherwise consume fewer units of paroxetine. Also, the patent term of the combination extends beyond that of paroxetine. Sales of paroxetine increase and the market for paroxetine is increased.

In another example, the market for paroxetine is increased by combining from about 0.5 g to about 20 g A-2497 and from about 10 mg to about 80 mg paroxetine. The resultant combination is consumed by individuals at risk of diarrhea due to paroxetine administration who otherwise consume fewer units of paroxetine. Also, the patent term of the combination extends beyond that of paroxetine. Sales of paroxetine increase and the market for paroxetine is increased.

In another example, the market for sertraline is increased by combining an amount of A-2497 therapeutically effective for the treatment, lessening, or prevention of diarrhea and an amount of sertraline therapeutically effective for the treatment, lessening, or prevention of depression. The resultant combination is consumed by individuals at risk of diarrhea due to sertraline administration who otherwise consume fewer units of sertraline. Also, the patent term of the combination extends beyond that of sertraline. Sales of sertraline increase and the market for sertraline is increased.

In another example, the market for sertraline is increased by combining from about 0.5 g to about 20 g A-2497 and from about 25 mg to about 200 mg sertraline. The resultant combination is consumed by individuals at risk of diarrhea due to sertraline administration who otherwise consume fewer units of sertraline. Also, the patent term of the combination extends beyond that of sertraline. Sales of sertraline increase and the market for sertraline is increased.

In another example, the market for the combination of amoxicllin and clavulinic acid is increased by combining an amount of A-2497 therapeutically effective for the treatment, lessening, or prevention of diarrhea and an amount of amoxicllin and clavulinic acid therapeutically effective for the treatment, lessening, or prevention of microbial infection. The resultant combination is consumed by individuals at risk of diarrhea due to amoxicllin and clavulinic acid administration who otherwise consume fewer units of amoxicllin and clavulinic acid. Also, the patent term of the combination extends beyond that of amoxicllin and clavulinic acid. Sales of amoxicllin and clavulinic acid increase and the market for amoxicllin and clavulinic acid is increased.

In another example, the market for omeprazole is increased by combining an amount of the copolymer of guanidine and polyethylenimine (also called “GUPEI”; disclosed in U.S. Pat. No. 6,040,315 to Day) and an amount of omeprazole which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of omeprazole. Also, the patent term of the combination extends beyond that of omeprazole. Sales of omeprazole increase and the market for omeprazole is increased.

In another example, the market for omeprazole is increased by combining from about 0.5 g to about 10 g GUPEI and from about 10 mg to about 50 mg omeprazole. The resultant combination is consumed by individuals who otherwise consume fewer units of omeprazole. Also, the patent term of the combination extends beyond that of omeprazole. Sales of omeprazole increase and the market for omeprazole is increased.

In another example, the market for ranitidine is increased by combining an amount of and an amount of ranitidine which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of ranitidine. Also, the patent term of the combination extends beyond that of ranitidine. Sales of ranitidine increase and the market for ranitidine is increased.

In another example, the market for ranitidine is increased by combining from about 0.5 g to about 10 g GUPEI and from about 50 mg to about 300 mg ranitidine. The resultant combination is consumed by individuals who otherwise consume fewer units of ranitidine. Also, the patent term of the combination extends beyond that of ranitidine. Sales of ranitidine increase and the market for ranitidine is increased.

In another example, the market for famotidine is increased by combining an amount of GUPEI and an amount of famotidine which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of famotidine. Also, the patent term of the combination extends beyond that of famotidine. Sales of famotidine increase and the market for famotidine is increased.

In another example, the market for famotidine is increased by combining from about 0.5 g to about 10 g GUPEI and from about 10 mg to about 40 mg famotidine. The resultant combination is consumed by individuals who otherwise consume fewer units of famotidine. Also, the patent term of the combination extends beyond that of famotidine. Sales of famotidine increase and the market for famotidine is increased.

In another example, the market for nizatidine is increased by combining an amount of GUPEI and an amount of nizatidine which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of nizatidine. Also, the patent term of the combination extends beyond that of nizatidine. Sales of nizatidine increase and the market for nizatidine is increased.

In another example, the market for nizatidine is increased by combining from about 0.5 g to about 10 g GUPEI and from about 75 mg to about 300 mg nizatidine. The resultant combination is consumed by individuals who otherwise consume fewer units of nizatidine. Also, the patent term of the combination extends beyond that of nizatidine. Sales of nizatidine increase and the market for nizatidine is increased.

In another example, the market for lansoprazole is increased by combining an amount of GUPEI and an amount of lansoprazole which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of lansoprazole. Also, the patent term of the combination extends beyond that of lansoprazole. Sales of lansoprazole increase and the market for lansoprazole is increased.

In another example, the market for lansoprazole is increased by combining from about 0.5 g to about 10 g GUPEI and from about 10 mg to about 120 mg lansoprazole. The resultant combination is consumed by individuals who otherwise consume fewer units of lansoprazole. Also, the patent term of the combination extends beyond that of lansoprazole. Sales of lansoprazole increase and the market for lansoprazole is increased.

In another example, the market for rabeprazole is increased by combining an amount of GUPEI and an amount of rabeprazole which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of ranitidine. Also, the patent term of the combination extends beyond that of rabeprazole. Sales of rabeprazole increase and the market for rabeprazole is increased.

In another example, the market for rabeprazole is increased by combining from about 0.5 g to about 10 g GUPEI and from about 10 mg to about 100 mg rabeprazole. The resultant combination is consumed by individuals who otherwise consume fewer units of rabeprazole. Also, the patent term of the combination extends beyond that of rabeprazole. Sales of rabeprazole increase and the market for rabeprazole is increased.

In another example, the market for pantoprazole is increased by combining an amount of GUPEI and an amount of pantoprazole which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of pantoprazole. Also, the patent term of the combination extends beyond that of pantoprazole. Sales of pantoprazole increase and the market for pantoprazole is increased.

In another example, the market for pantoprazole is increased by combining from about 0.5 g to about 10 g GUPEI and from about 20 mg to about 80 mg pantoprazole. The resultant combination is consumed by individuals who otherwise consume fewer units of pantoprazole. Also, the patent term of the combination extends beyond that of pantoprazole. Sales of pantoprazole increase and the market for pantoprazole is increased.

In another example, the market for esomeprazole is increased by combining an amount of GUPEI and an amount of esomeprazole which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of esomeprazole. Also, the patent term of the combination extends beyond that of esomeprazole. Sales of esomeprazole increase and the market for esomeprazole is increased.

In another example, the market for esomeprazole is increased by combining from about 0.5 g to about 10 g GUPEI and from about 10 mg to about 80 mg esomeprazole. The resultant combination is consumed by individuals who otherwise consume fewer units of esomeprazole. Also, the patent term of the combination extends beyond that of esomeprazole. Sales of esomeprazole increase and the market for esomeprazole is increased.

In another example, the market for cimetidine is increased by combining an amount of GUPEI and an amount of cimetidine which together are therapeutically effective for the relief of duodenal ulcer, gastric ulcer, gastroesophageal reflux disease, esophagitis, or pathological hypersecretory condition. The resultant combination is consumed by individuals who otherwise consume fewer units of cimetidine. Also, the patent term of the combination extends beyond that of cimetidine. Sales of cimetidine increase and the market for cimetidine is increased.

In another example, the market for cimetidine is increased by combining from about 0.5 g to about 10 g GUPEI and from about 100 mg to about 1600 mg cimetidine. The resultant combination is consumed by individuals who otherwise consume fewer units of cimetidine. Also, the patent term of the combination extends beyond that of cimetidine. Sales of cimetidine increase and the market for cimetidine is increased.

In another example, the market for a non-steroidal inflammatory agent (NSAID) is increased by combining an amount of GUPEI and an amount of NSAID which together are therapeutically effective for the relief of inflammation, fever or pain, but which together give rise to less adverse gastrointestinal side effect than the same amount of NSAID without GUPEI. The resultant combination is consumed by individuals at risk of adverse side effect due to NSAID administration who otherwise consume fewer units of NSAID. Also, the patent term of the combination extends beyond that of NSAID. Sales of NSAID increase and the market for NSAID is increased.

In another example, the market for NSAID is increased by combining from about 0.5 g to about 10 g GUPEI and from about 25 mg to about 400 mg sulindac, from about 125 mg to about 1500 mg diflunisal, from about 6.25 mg to about 75 mg rofecoxib, from about 20 mg to about 300 mg diclofenac, from about 100 mg to about 2000 mg tolmentin, from about 50 mg to about 500 mg celecoxib, from about 5 mg to about 50 mg piroxicam, from about 100 mg to about 2000 mg nabumetone, or from about 100 mg to about 1000 mg etodolac. The resultant combination is consumed by individuals at risk of adverse side effect due to NSAID administration who otherwise consume fewer units of NSAID. Also, the patent term of the combination extends beyond that of NSAID. Sales of NSAID increase and the market for NSAID is increased.

In another example, the market for HMGCoA reductase inhibitor is increased by combining an amount of A-2497 therapeutically effective for the treatment, lessening, or prevention of hypercholesterolemia and an amount of simvastatin, lovastatin, atorvastatin, fluvastatin, pravastatin, cerivastatin, or rosuvastatin therapeutically effective for the treatment, lessening, or prevention of hypercholesterolemia. The resultant combination is consumed by individuals at risk of hypercholesterolemia. The patent term of the combination extends beyond that of the HMGCoA reductase inhibitor. Sales of the HMGCoA reductase inhibitor increase and the market for the HMGCoA reductase inhibitor is increased.

In another example, the market for antimicrobial agent is increased by combining an amount of A-2497 therapeutically effective for the treatment, lessening, or prevention of diarrhea and an amount of ciprofloxacin, cefuroxime axetil, fluconazole, clarithromycin, cefprozil, azithromycin, or terbinafine therapeutically effective for the treatment, lessening, or prevention of microbial infection. The resultant combination is consumed by individuals at risk of diarrhea due to antimicrobial agent administration who otherwise consume fewer units of antimicrobial agent. Also, the patent term of the combination extends beyond that of antimicrobial agent. Sales of antimicrobial agent increase and the market for antimicrobial agent is increased.

In another example, the market for olsalazine is increased by combining an amount of A-2497 therapeutically effective for the treatment, lessening, or prevention of diarrhea and an amount of olsalazine therapeutically effective for the treatment of ulcerative colitis. The resultant combination is consumed by individuals at risk of diarrhea due to olsalazine administration who otherwise consume fewer units of olsalazine. Also, the patent term of the combination extends beyond that of olsalazine. Sales of olsalazine increase and the market for olsalazine is increased.

In another example, the market for alosetron is increased by combining an amount of A-2497 therapeutically effective for the treatment, lessening, or prevention of irritable bowel syndrome and an amount of alosetron therapeutically effective for the treatment, lessening, or prevention of diarrhea-predominant irritable bowel syndrome. The resultant combination possesses greater efficacy than alosetron for the treatment, lessening, or prevention of irritable bowel syndrome and is consumed by individuals at risk of constipation due to alosetron administration who otherwise consume fewer units of alosetron. Also, the patent term of the combination extends beyond that of alosetron. Sales of alosetron increase and the market for alosetron is increased.

In another example, the market for alosetron is increased by combining from about 0.5 g to about 20 g A-2497 and from about 0.5 mg to about 2 mg alosetron. The resultant combination possesses greater efficacy than alosetron for the treatment, lessening, or prevention of irritable bowel syndrome and is consumed by individuals at risk of constipation due to alosetron administration who otherwise consume fewer units of alosetron. Also, the patent term of the combination extends beyond that of alosetron. Sales of alosetron increase and the market for alosetron is increased.

In another example, the market for ondansetron is increased by combining an amount of A-2497 therapeutically effective for the treatment, lessening, or prevention of irritable bowel syndrome and an amount of ondansetron therapeutically effective for the treatment, lessening, or prevention of nausea and vomiting. The resultant combination is consumed by individuals at risk of constipation due to ondansetron administration who otherwise consume fewer units of ondansetron. Also, the patent term of the combination extends beyond that of ondansetron. Sales of ondansetron increase and the market for ondansetron is increased.

In another example, the market for ondansetron is increased by combining from about 0.5 g to about 20 g A-2497 and from about 2 mg to about 20 mg ondansetron. The resultant combination is consumed by individuals at risk of constipation due to ondansetron administration who otherwise consume fewer units of ondansetron. Also, the patent term of the combination extends beyond that of ondansetron. Sales of ondansetron increase and the market for ondansetron is increased.

In another example, the market for tegaserod is increased by combining an amount of A-2497 therapeutically effective for the treatment, lessening, or prevention of irritable bowel syndrome and an amount of tegaserod therapeutically effective for the treatment, lessening, or prevention of irritable bowel syndrome. The resultant combination possesses greater efficacy than tegaserod for the treatment, lessening, or prevention of irritable bowel syndrome and is consumed by individuals who otherwise consume fewer units of tegaserod. Also, the patent term of the combination extends beyond that of tegaserod. Sales of tegaserod increase and the market for tegaserod is increased.

In another example, the market for tegaserod is increased by combining from about 0.5 g to about 20 g A-2497and from about 0.5 mg to about 5 mg tegaserod. The resultant combination possesses greater efficacy than tegaserod for the treatment, lessening, or prevention of irritable bowel syndrome and is consumed by individuals who otherwise consume fewer units of tegaserod. Also, the patent term of the combination extends beyond that of tegaserod. Sales of tegaserod increase and the market for tegaserod is increased.

In another example, the market for leflunomide is increased by combining an amount of A-2497 therapeutically effective for the treatment, lessening, or prevention of diarrhea and an amount of leflunomide therapeutically effective for the treatment of rheumatoid arthritis. The resultant combination is consumed by individuals at risk of diarrhea due to leflunomide administration who otherwise consume fewer units of leflunomide. Also, the patent term of the combination extends beyond that of leflunomide. Sales of leflunomide increase and the market for leflunomide is increased.

In another example, the market for leflunomide is increased by combining from about 0.5 g to about 20 g A-2497 and from about 10 mg to about 200 mg leflunomide. The resultant combination is consumed by individuals at risk of diarrhea due to leflunomide administration who otherwise consume fewer units of leflunomide. Also, the patent term of the combination extends beyond that of leflunomide. Sales of leflunomide increase and the market for leflunomide is increased.

In another example, the market for orlistat is increased by combining an amount of A-2497 therapeutically effective for the treatment, lessening, or prevention of diarrhea or fecal urgency and an amount of orlistat therapeutically effective for the treatment of obesity. The resultant combination is consumed by individuals at risk of diarrhea or fecal urgency due to orlistat administration who otherwise consume fewer units of orlistat. Also, the patent term of the combination extends beyond that of orlistat. Sales of orlistat increase and the market for orlistat is increased.

In another example, the market for metformin is increased by combining an amount of A-2497 therapeutically effective for the treatment, lessening, or prevention of diarrhea and an amount of metformin therapeutically effective for the treatment of hyperglycemia. The resultant combination is consumed by individuals at risk of diarrhea due to metformin administration who otherwise consume fewer units of metformin. Also, the patent term of the combination extends beyond that of metformin. Sales of metformin increase and the market for metformin is increased.

In another example, the market for glyburide and metformin is increased by combining an amount of A-2497 therapeutically effective for the treatment, lessening, or prevention of diarrhea and an amount of glyburide and metformin therapeutically effective for the treatment of hyperglycemia. The resultant combination is consumed by individuals at risk of diarrhea due to glyburide and metformin administration who otherwise consume fewer units of glyburide and metformin. Also, the patent term of the combination extends beyond that of glyburide and metformin. Sales of glyburide and metformin increase and the market for glyburide and metformin is increased.

In another example, the market for a drug previously formulated in a dosage form containing gelatin is increased by combining the drug with A-2497 or GUPEI in a dosage form lacking gelatin. The resultant combined dosage form is consumed by individuals eschewing gelatin who otherwise consume fewer units of the drug. Also, the patent term of the resultant combined dosage form extends beyond that of the drug. Sales of the drug increase and the market for the drug is increased.

It is to be understood that the invention is not to be limited to the exact details of operation, or to the exact compositions, methods, procedures, or embodiments shown and described, as obvious modifications and variations will be apparent to one skilled in the art, and the invention is therefore to be limited only by the full scope which can fairly, legally, or equitably be accorded to the appended claims.

I claim:

1. A method of increasing the market for an existing pharmaceutical, said method comprising the step of making a reformulated pharmaceutical by combining the existing pharmaceutical with a nondegradable synthetic polymer that is not substantially absorbed from the human alimentary canal.

2. The method of claim 1 wherein the existing pharmaceutical is a selective serotonin reuptake inhibitor, a proton pump inhibitor, an H2 receptor antagonist, an HMGCoA reductase inhibitor, an antimicrobial agent, an NSAID, olsalazine, alosetron, ondansetron, tegaserod, leflunomide, orlistat, metformin, or the combination of glyburide and metformin.

3. The method of claim 1 wherein the nondegradable polymer is A-2497 or GUPEI.

4. The method of claim 1 comprising the additional step of achieving a sale of the reformulated pharmaceutical to an individual at risk of experiencing adverse side effect from the existing pharmaceutical.

5. The method of claim 1 comprising the additional step of achieving a sale of the reformulated pharmaceutical to an individual who wishes to purchase a pharmaceutical that possesses greater efficacy than the efficacy possessed by the existing pharmaceutical.

6. The method of claim 1 comprising the additional step of gaining for the reformulated pharmaceutical a patent term greater in temporal or geographic scope than that of the existing pharmaceutical.

7. The method of claim 1 comprising the additional step of achieving a sale of the reformulated pharmaceutical to an individual who does not consume the existing pharmaceutical due to the presence of a substance contained in the existing pharmaceutical but present in smaller quantity in, or absent from, the reformulated pharmaceutical.

8. The method of claim 2 wherein the selective serotonin reuptake inhibitor is fluoxetine, paroxetine, or sertraline.

9. The method of claim 2 wherein the proton pump inhibitor is omeprazole, lansoprazole, rabeprazole, pantoprazole, or esomeprazole.

10. The method of claim 2 wherein the H2 receptor antagonist is nizatidine, famotidine, cimetidine, or ranitidine.

11. The method of claim 2 wherein the HMGCoA reductase inhibitor is simvastatin, lovastatin, atorvastatin, fluvastatin, pravastatin, cerivastatin, or rosuvastatin.

12. The method of claim 2 wherein the antimicrobial agent is ciprofloxacin, cefuroxime axetil, fluconazole, clarithromycin, cefprozil, azithromycin, terbinafine, or the combination of amoxicillin and clavulinic acid.

13. The method of claim 2 wherein the NSAID is sulindac, diflunisal, rofecoxib, diclofenac, tolmentin, celecoxib, piroxicam, nabumetone, or etodolac.

14. The method of claim 3 wherein the existing pharmaceutical is a selective serotonin reuptake inhibitor, a proton pump inhibitor, an H2 receptor antagonist, an HMGCoA reductase inhibitor, an antimicrobial agent, an NSAID, olsalazine, alosetron, ondansetron, tegaserod, leflunomide, orlistat, metformin, or the combination of glyburide and metformin.

15. The method of claim 3 wherein the selective serotonin reuptake inhibitor is fluoxetine, paroxetine, or sertraline.

16. The method of claim 3 wherein the proton pump inhibitor is omeprazole, lansoprazole, rabeprazole, pantoprazole, or esomeprazole.

17. The method of claim 3 wherein the H2 receptor antagonist is nizatidine, famotidine, cimetidine, or ranitidine.

18. The method of claim 3 wherein the HMGCoA reductase inhibitor is simvastatin, lovastatin, atorvastatin, fluvastatin, pravastatin, cerivastatin, or rosuvastatin.

19. The method of claim 3 wherein the antimicrobial agent is ciprofloxacin, cefuroxime axetil, fluconazole, clarithromycin, cefprozil, azithromycin, terbinafine, or the combination of amoxicillin and clavulinic acid.

20. The method of claim 3 wherein the NSAID is sulindac, diflunisal, rofecoxib, diclofenac, tolmentin, celecoxib, piroxicam, nabumetone, or etodolac.

21. The method of claim 3 comprising an additional step chosen from the group consisting of: (a) achieving a sale of the reformulated pharmaceutical to an individual at risk of experiencing adverse side effect from the existing pharmaceutical; (b) achieving a sale of the reformulated pharmaceutical to an individual who wishes to purchase a pharmaceutical that possesses greater efficacy than the efficacy possessed by the existing pharmaceutical; (c) gaining for the reformulated pharmaceutical a patent term greater in temporal or geographic scope than that of the existing pharmaceutical; and (d) achieving a sale of the reformulated pharmaceutical to an individual who does not consume the existing pharmaceutical due to the presence of a substance contained in the existing pharmaceutical but present in smaller quantity in, or absent from, the reformulated pharmaceutical.
